SEQUENCE LISTING (43 sequences)

SEQ ID NO: 1 - protein, 15 aa, Artificial Sequence
  A synthetic peptide
XXYXXYXXXYYXXXX

SEQ ID NO: 2 - protein, 13 aa, Artificial Sequence
  A synthetic peptide
XXYXXYXXXYYYX

SEQ ID NO: 3 - protein, 15 aa, Artificial Sequence
  A synthetic peptide
GDYADYDGGYYYDMD

SEQ ID NO: 4 - protein, 14 aa, Artificial Sequence
  A synthetic peptide
DYADYDGGYYYDMD

SEQ ID NO: 5 - protein, 15 aa, Artificial Sequence
  A synthetic peptide
GDYADYDGGYYYDGG

SEQ ID NO: 6 - protein, 14 aa, Artificial Sequence
  A synthetic peptide
DYADYDGGYYYDGG

SEQ ID NO: 7 - protein, 24 aa, Artificial Sequence
  A synthetic peptide
NSIAGVAAAGDYADYDGGYYYDMD

SEQ ID NO: 8 - protein, 12 aa, Artificial Sequence
  A synthetic peptide
GDYADYDGGYYY

SEQ ID NO: 9 - protein, 6 aa, Artificial Sequence
  A synthetic peptide
DYYYPD

SEQ ID NO: 10 - protein, 6 aa, Artificial Sequence
  A synthetic peptide
YLDYYY

SEQ ID NO: 11 - protein, 10 aa, Artificial Sequence
  A synthetic peptide
ENYSYDLDYY

SEQ ID NO: 12 - protein, 13 aa, Artificial Sequence
  A synthetic peptide
GDYADYDGGYYYD

SEQ ID NO: 13 - protein, 14 aa, Artificial Sequence
  A synthetic peptide
GDYADYDGGYYYDM

SEQ ID NO: 14 - protein, 14 aa, Artificial Sequence
  A synthetic peptide
GDYADYDGGYYYDG

SEQ ID NO: 15 - protein, 12 aa, Artificial Sequence
  A synthetic peptide
DYADYDGGYYYD

SEQ ID NO: 16 - protein, 12 aa, Artificial Sequence
  A synthetic peptide
DYADYDGGYYYD

SEQ ID NO: 17 - protein, 15 aa, Artificial Sequence
  A synthetic peptide
DYADYDGGYYYDMDG

SEQ ID NO: 18 - protein, 236 aa, Artificial Sequence
  A synthetic peptide
AADPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 711 bp, Artificial Sequence
  A synthetic oligonucleotide
gccgcggatcccgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 20 - protein, 178 aa, Artificial Sequence
  A synthetic polypeptide
KKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLA

SEQ ID NO: 21 - DNA, 533 bp, Artificial Sequence
  A synthetic oligonucleotide
aagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaagaaacttccccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctggct

SEQ ID NO: 22 - protein, 11 aa, Artificial Sequence
  A synthetic polypeptide
XXYXXYXXXYY

SEQ ID NO: 23 - protein, 4 aa, Artificial Sequence
  A synthetic polypeptide
GSGG

SEQ ID NO: 24 - protein, 8 aa, Artificial Sequence
  A synthetic polypeptide
GSGGGSGG

SEQ ID NO: 25 - protein, 12 aa, Artificial Sequence
  A synthetic polypeptide
GSGGGSGGGSGG

SEQ ID NO: 26 - protein, 16 aa, Artificial Sequence
  A synthetic polypeptide
GSGGGSGGGSGGGSGG

SEQ ID NO: 27 - protein, 20 aa, Artificial Sequence
  A synthetic polypeptide
GSGGGSGGGSGGGSGGGSGG

SEQ ID NO: 28 - protein, 24 aa, Artificial Sequence
  A synthetic polypeptide
GSGGGSGGGSGGGSGGGSGGGSGG

SEQ ID NO: 29 - protein, 28 aa, Artificial Sequence
  A synthetic polypeptide
GSGGGSGGGSGGGSGGGSGGGSGGGSGG

SEQ ID NO: 30 - protein, 32 aa, Artificial Sequence
  A synthetic polypeptide
GSGGGSGGGSGGGSGGGSGGGSGGGSGGGSGG

SEQ ID NO: 31 - protein, 36 aa, Artificial Sequence
  A synthetic polypeptide
GSGGGSGGGSGGGSGGGSGGGSGGGSGGGSGGGSGG

SEQ ID NO: 32 - protein, 40 aa, Artificial Sequence
  A synthetic polypeptide
GSGGGSGGGSGGGSGGGSGGGSGGGSGGGSGGGSGGGSGG

SEQ ID NO: 33 - protein, 4 aa, Artificial Sequence
  A synthetic polypeptide
GDGG

SEQ ID NO: 34 - protein, 8 aa, Artificial Sequence
  A synthetic polypeptide
GDGGGDGG

SEQ ID NO: 35 - protein, 12 aa, Artificial Sequence
  A synthetic polypeptide
GDGGGDGGGDGG

SEQ ID NO: 36 - protein, 16 aa, Artificial Sequence
  A synthetic polypeptide
GDGGGDGGGDGGGDGG

SEQ ID NO: 37 - protein, 20 aa, Artificial Sequence
  A synthetic polypeptide
GDGGGDGGGDGGGDGGGDGG

SEQ ID NO: 38 - protein, 24 aa, Artificial Sequence
  A synthetic polypeptide
GDGGGDGGGDGGGDGGGDGGGDGG

SEQ ID NO: 39 - protein, 28 aa, Artificial Sequence
  A synthetic polypeptide
GDGGGDGGGDGGGDGGGDGGGDGGGDGG

SEQ ID NO: 40 - protein, 32 aa, Artificial Sequence
  A synthetic polypeptide
GDGGGDGGGDGGGDGGGDGGGDGGGDGGGDGG

SEQ ID NO: 41 - protein, 36 aa, Artificial Sequence
  A synthetic polypeptide
GDGGGDGGGDGGGDGGGDGGGDGGGDGGGDGGGDGG

SEQ ID NO: 42 - protein, 40 aa, Artificial Sequence
  A synthetic polypeptide
GDGGGDGGGDGGGDGGGDGGGDGGGDGGGDGGGDGGGDGG

SEQ ID NO: 43 - protein, 12 aa, Artificial Sequence
  A synthetic polypeptide
DYADYDGGYYYD

(19) United States
(12) Patent Application Publication
(10) Pub. No.: US 2011/0305670 A1
(43) Pub. Date: Dec. 15, 2011

(54) NUCLEIC ACID ENCODING FUSION POLYPEPTIDES THAT PREVENT OR INHIBIT HIV INFECTION

(75) Inventor: Michael R. Farzan, Brookline, MA (US)

(73) Assignee: President and Fellows of Harvard College, Cambridge, MA (US)

(21) Appl. No.: 12/813,249

(22) Filed: Jun. 10, 2010

(51) Int. Cl. A61K 35/76; A61P 31/12; A61K 31/7052; C12N 15/13; C12N 7/01

(52) U.S. Cl. 424/93.2; 536/23.4; 435/235.1; 514/44 R

(57) ABSTRACT

The present invention is directed to an isolated nucleic acid molecule encoding a fusion polypeptide the expression of which in cells is capable of blocking the entry of HIV-1 into host cells and methods of using the nucleic acid molecule.

### STATEMENT OF GOVERNMENT FUNDING

The invention described herein was made with Government support under NIH Grant No. R01 AI48425 awarded by the Department of Health and Human Services. The Government therefore has certain rights in the invention.

### FIELD OF THE INVENTION

The present invention is directed to a nucleic acid molecule encoding a fusion polypeptide that can be used to prevent or inhibit the binding of gp120 to receptors on the surface of immune cells. By preventing this interaction, the fusion polypeptide can be used to inhibit or block entry of HIV into cells.

### BACKGROUND

The HIV virus responsible for causing AIDS enters immune cells through a multi-step process (Berger, AIDS, 11:S3 (1997); Doranz, et al, Immunol. Res., 16:15 (1997)). Initially, gp120 located on the HIV viral surface binds to a CD4 receptor on the surface of the host cell. This causes the gp120 protein to undergo a conformational change that allows it to bind to a second cell surface receptor, CCR5 (Dragic, et al., Nature, 381:667 (1996); Deng, et al., Nature, 381:661 (1996)). It is this second binding step that ultimately leads to membrane fusion and viral entry.

Biochemical studies have revealed that a portion of the CCR5 receptor near its amino terminus is critical for interaction with gp120 and that there are several sulfated tyro sines in this region that are essential for binding (Farzan, et al, J. Virol., 72:1160 (1998); Farzan, et al., Cell, 96:667 (1999)). Attempts have been made to model peptides based upon the CCR5 binding region and use them to block the entry of HIV into immune cells (Farzan, et al., J. Biol. Chem., 275:33516 (2000); Cormier, et al., Proc. Nat'l Acad. Sci., 97:5762 (2000)). However, the peptides that have been developed thus far appear to have relatively low affinity for gp120 and this may ultimately limit their clinical usefulness.

In an alternative approach, researchers have attempted to make antibodies against gp120 that block the entry of HIV into host cells (see, e.g., Cole, et al., Virology, 290:59 (2001); Fouts, et al., J. Virol., 71:2779 (1997); Ho, et al., J. Virol., 65:489 (1991)). Although antibodies of this type have a high affinity for antigen, HIV-1 can readily escape from any neutralizing antibody because the surface of interaction necessarily includes unconserved regions of gp120.

### SUMMARY OF THE INVENTION

The invention provides an inhibitor of HIV-1 replication with one or more of the following properties: (1) it neutralizes most HIV-1 isolates, and imposes a high fitness cost on viruses that escape, (2) it stably suppresses viral replication to the point where subsequent transmission is unlikely, (3) it has no significant side effects or toxicity, (4) host immunity does not interfere with its long-term expression, and/or (5) it is efficiently expressed following a single intramuscular injection. The most challenging remaining hurdle to a therapeutic molecule in an ongoing HIV-1 infection is the problem of viral escape. As described hereinbelow, receptor/coreceptor analogues like e3-CD4-Ig and e5-CD4-Ig are uniquely suited to impose a high fitness cost on escaping viruses. In contrast, anti-gp120 antibodies bind a broader surface of the envelope glycoprotein, permitting easy escape through alteration of a non-conserved amino acid.

The invention thus provides isolated nucleic acid molecules (polynucleotides) encoding a fusion (chimeric) polypeptide having a CCR5 binding peptide (a CCR5 mimetic peptide which binds HIV-1 gp120 by mimicking the HIV-1 coreceptor CCR5), a CD4 polypeptide that binds gp120 and a Fc binding region of an immunoglobulin (Ig). In one embodiment, the CD4 polypeptide is a soluble CD4 polypeptide, e.g., one that includes domains 1 and 2 of CD4. In one embodiment, the sequence of the CD4 polypeptide has one or more substitutions relative to the corresponding sequence of a wild-type CD4 (it is a “variant” CD4 polypeptide). In one embodiment, the fusion polypeptide comprises sequences from the light chain of an immunoglobulin fused to those from a heavy chain (a scF). The immunoglobulin portion of the fusion polypeptide may be any isotype of immunoglobulin, e.g., IgG, IgM, IgA, IgE or IgD, or any subtype of an immunoglobulin, e.g., IgG1, IgG2, IgG3, IgG4, or IgG5. In one embodiment, the fusion polypeptide of the invention includes a CCR5 mimetic peptide, for instance, a twelve-amino-acid CCR5 mimetic peptide, that, when fused to CD4-Ig (e3-CD4-Ig), e.g., via a peptide linker, neutralizes a range of clade B HIV-1 isolates up to fifty times more efficiently than unmodified CD4-Ig, with IC₅₀s in the picomolar range. In one embodiment, the fusion polypeptide of the invention includes a twelve-amino-acid CCR5-binding peptide that, when fused to a variant soluble CD4 polypeptide, e.g., e5-CD4-Ig, a variant CD4 with a substitution at position 40 such as a Q40A substitution, further enhances the on-rate of the fusion polypeptide. e5-CD4-Ig also neutralizes HIV-1 replication more broadly and potently than the highest affinity and most potent neutralizing antibodies. There are likely several reasons for this enhanced potency: (1) the CD4 and CCR5-mimetic components of e5-CD4-Ig both cooperate to enhance the affinity of the other for the HIV-1 envelope glycoprotein, (2) in contrast to CD4-Ig, a single e5-CD4-Ig molecule binds more than one monomer of the envelope glycoprotein trimer, (3) e5-CD4-Ig may inactivate the envelope glycoprotein more effectively than CD4-Ig, (4) the affinity of the modified CD4 domain is higher for gp120 than the native CD4, and (5) at low concentrations, CD4-Ig actually enhances infection, whereas e5-CD4-Ig does not.

The invention thus provides a therapeutic composition comprising an amount of a nucleic acid molecule encoding, or an amount of, a fusion polypeptide comprising a CCR5 mimetic peptide, a soluble CD4 polypeptide that binds gp120 and a Fc binding region of an immunoglobulin, which upon administration is effective to prevent, inhibit or treat HIV infection in a mammal, e.g., a human. In one embodiment, the Fc binding region is a Fc binding region from IgG1, IgG2 or IgA. In one embodiment, the Fc binding region has at least 80%, 85%, 90%, 95%, 99% or 100% amino acid sequence identity to a polypeptide having SEQ ID NO:18, or a portion thereof that binds Fc. In one embodiment, the soluble portion of CD4 includes domains 1 and 2 of CD4, e.g., residues 1 to about 178 of CD4. In one embodiment, the soluble portion of CD4 has at least 80%, 85%, 90%, 95%, 99% or 100% amino acid sequence identity to a polypeptide having SEQ ID NO:20, or a portion thereof that binds HIV gp120. In one embodiment, the fusion polypeptide of the invention has enhanced affinity for HIV gp120 relative to a chimeric protein having sequences corresponding to wild-type CD4 and the Fc binding region or a chimeric protein having sequences corresponding to the CCR5 mimetic peptide and the Fc binding region.

In one embodiment, the CCR5 mimetic peptide is from 10 to 35 amino acids in length and comprises X₁X₂(Y)X₃X₄(Y)X₅X₆X₇(Y)X₈X₉X₁₀X₁₁ (SEQ ID NO:1), wherein X₁-X₂ and X₈-X₁₁ are independently absent or independently D, N, Q, H, G, Y, M, K, T, S, E, P or sY, wherein sY indicates a sulfated tyrosine, wherein X₃-X₄ and X₅-X₇ are independently D, N, Q, H, G, Y, M, K, T, S, E, P or sY, and wherein each (Y) may be sulfated. In one embodiment, the CCR5 mimetic peptide is from 10 to 35 amino acids in length, and comprises X₁X₂YX₃X₄YX₅X₆X₇YYYX₈ (SEQ ID NO:2), wherein X₁ is absent or is G, A, L, or I; X₂, X₄ and X₅ are independently D, N, Q, H, or E; X₃, X₆ and X₇ are independently G, A, L, or I; and X₈ is D, N, H, Q, or E. In one embodiment, the CCR5 mimetic peptide is from 10 to 35 amino acids in length, and comprises X₁X₂YX₃X₄YX₅X₆X₇YY (SEQ ID NO:18), wherein X₁ is absent or is G, A, L, or I; X₂ is absent or is D, N, Q, H, or E; X₄ and X₅ are independently D, N, Q, H, or E; and X₃, X₆ and X₇ are independently G, A, L, or I. In one embodiment, the CCR5 mimetic peptide is C-terminal to the Fc binding region which itself is C-terminal to the CD4 polypeptide. A peptide linker may be placed between any of the CCR5 mimetic peptide, the CD4 polypeptide that binds gp120 and the Fc binding region of an immunoglobulin. A peptide linker when present in a fusion polypeptide of the invention does not substantially alter, e.g., reduce or inhibit, the activity of any of the CCR5 mimetic peptide, a soluble CD4 polypeptide that binds gp120 or a Fc binding region of an immunoglobulin and also, optionally, has no or reduced immunogenicity (e.g., does not elicit a sequence specific immune response). In one embodiment, the peptide linker is from 1 to 50 amino acids in length, e.g., any integer between 1 to 50, for instance, from 3 to 15 amino acids, or any integer in between 3 to 15, in length. A peptide linker may include a plurality of G, D and/or S residues, or a combination thereof. In one embodiment, the peptide linker includes at least two G residues. In one embodiment, the peptide linker includes (GSGG)ₓ or (GDGG)ₓ, wherein x is 1 to 10, e.g., 1 to 5 (SEQ ID NO:17).

In one embodiment, one or more of the tyro sines in the CCR5 mimetic peptide or the fusion polypeptide are sulfated, or are replaced with tyrosine sulfate or phenylalanine methyl sulfatate. In one embodiment at least two or three tyrosines in the CCR5 mimetic peptide or the fusion polypeptide are sulfated.

In one embodiment, the invention provides a therapeutic composition comprising an amount of a nucleic acid molecule encoding a fusion polypeptide comprising a CCR5 mimetic peptide, a soluble CD4 polypeptide that binds gp120 and a Fc binding region of an immunoglobulin, which upon administration is effective to prevent, inhibit or treat HIV infection in a primate. In one embodiment, the fusion polypeptide has enhanced affinity for HIV gp120 relative to a chimeric protein having sequences corresponding to wild-type CD4 and the Fc binding region or a chimeric protein having sequences corresponding to the CCR5 mimetic peptide and the Fc binding region. In one embodiment, the CCR5 mimetic peptide is from 10 to 35 amino acids in length and comprises X₁X₂(sY)X₃X₄(sY)X₅X₆X₇(sY)(sY)X₈X₉X₁₀X₁₁ (SEQ ID NO:1) wherein X₁-X₂ and X₈-X₁₁ are independently absent or independently D, N, Q, H, G, Y, M, K, T, S, E, P or sY, wherein sY indicates a sulfated tyrosine, wherein X₃-X₄ and X₅-X₇ are independently D, N, Q, H, G, Y, M, K, T, S, E, P or sY, and wherein each (Y) may be sulfated. In one embodiment, the CCR5 mimetic peptide is from 10 to 35 amino acids in length, and X₁X₂YX₃X₄YX₅X₆X₇YYYX₈ (SEQ ID NO:2), wherein X₁ is absent or is G, A, L, or I; X₂, X₄ and X₅ are independently D, N, Q, H, or E; X₃, X₆ and X₇ are independently G, A, L, or I; and X₈ is D, N, H, Q, or E. In one embodiment, the CCR5 mimetic peptide is from 10 to 35 amino acids in length, and comprises X₁X₂YX₃X₄YX₅X₆X₇YY (SEQ ID NO:18), wherein Xᵢ is absent or is G, A, L, or I; X₂ is absent or is D, N, Q, H, or E; X₄ and X₅ are independently D, N, Q, H, or E; and X₃, X₆ and X₇ are independently G, A, L, or I. In one embodiment, the CCR5 mimetic peptide has GDYADYDGGYYYDMD (SEQ ID NO:3), DYADYDGGYYYDMD (SEQ ID NO:4), GDYADYDGGYYYDGG (SEQ ID NO:5), DYADYDGGYYYDGG (SEQ ID NO:6), NSIAGVAAAGDYADYDGGYYYDMD (SEQ ID NO:7) or GDYADYDGGYYY (SEQ ID NO:8) or includes DYYYPD (SEQ ID NO:9), YLDYYY (SEQ ID NO:10), DYADYDGGYYYD (SEQ ID NO:16) or ENYSYDLDYY (SEQ ID NO:11). A peptide linker may be placed between any of the CCR5 mimetic peptide, the soluble CD4 binding polypeptide that binds gp120 and the Fc binding region of an immunoglobulin. In one embodiment, the peptide linker is from 1 to 50 amino acids in length, e.g., any integer between 1 to 50. In one embodiment, the peptide linker includes GSGG.

It will be recognized by those of skill in the art that conservative amino acid substitutions, e.g., substituting one acidic or basic amino acid for another, can often be made without affecting the biological activity of a peptide or protein. Minor variations in sequence of this nature may be made in any of the peptides disclosed herein, provided that these changes do not substantially reduce (e.g., by 15% or more) the ability of the peptide or fusion polypeptide to neutralize the entry of HIV-1 into immune cells. In one embodiment, the CCR5 mimetic peptide has an amino acid sequence having at least 80%, 85%, 90% or 95% amino acid sequence identity to one of SEQ ID NOs: 1-16 or 18. One, some or all of the tyrosines may be sulfated and other tyrosine residues present in fusion polypeptide may, optionally, also be sulfated. As discussed above, the sulfated tyrosines may be replaced with either tyrosine sulfonate or phenylalanine methyl sulfatate. Neutralization may be tested using methods known to the art, e.g., for measuring the entry of virus into cells.

A composition of the invention may include the nucleic acid molecule of the invention, e.g., in a viral delivery vehicle. Thus, in one embodiment, the invention provides a recombinant virus, for instance a recombinant adeno-associated virus (AAV), comprising a nucleic acid molecule encoding a fusion polypeptide comprising a CCR5 mimetic peptide, a CD4 polypeptide that binds gp120 and a Fc binding region of an immunoglobulin. In one embodiment, the recombinant virus is a recombinant AAV, e.g., self-complementary adeno-associated virus (scAAV) vectors, that provides for safe, unobtrusive and sustained expression (>2 year) of high levels of protein therapeutics. In one embodiment, the recombinant viruses of the invention are useful in late stage infections, e.g., the treatment of HIV infected patients.

The nucleic acid molecules, recombinant viruses or fusion polypeptide of the invention are useful in compositions to prevent, inhibit or treat HIV infection in a mammal. The nucleic acid molecules, recombinant viruses or fusion polypeptides described herein may be either used alone or in conjunction with other agents that are useful in prophylaxis and/or treatment of the HIV-1 virus. These nucleic acid molecules may be incorporated into an expression vector in which it is operably linked to a promoter. The expression vector may be used to transform a host cell which can then produce the fusion polypeptide in vitro or in vivo. Once made in vitro, the fusion polypeptide can be isolated. The invention thus includes expression vectors in which the nucleic acid molecule is operably linked to a promoter, and host cells transformed with the expression vector.

### BRIEF DESCRIPTION OF THE DRAWINGS

FIG. 1. CCR5 mimetic peptide (CCR5-mim) fused to Ig. CCR5mim-Ig associates with two gp120 monomers of the HIV-1 envelope glycoprotein trimer. Binding of a CD4mim-Ig and the CCR5mim-Ig to the HIV-1 envelope glycoprotein are compared. A) Precipitation of radiolabeled monomeric gp120 (ADA isolate) by CD4mim-Ig and CCR5mim-Ig, indicating higher affinity by the former peptide. B) Binding of each peptide-Ig to cell-expressed trimeric ADA envelope glycoprotein, measured by flow cytometry and indicating comparable binding of these constructs to the trimer. C) Experiment as in B) except that several peptide-Ig concentrations are assayed and a control Fc-doman (“Ig only”) is included. Crossing of the binding curves is repeatable and explained in the text. D) The structure of the gp120/412d/CD4 complex is fitted to a cryoEM-obtained density of the envelope glycoprotein bound to CD4 and 17b (Liu et al., Nature, 455:109 (2008)). Only the tyrosine-sulfated heavy-chain CDR3s of 412d are shown (green). Note their proximity in the trimer. In contrast, the distance between C-termini of CD4-d1d2 (red) is too great for CD4-Ig to bind more than one gp120 monomer.

FIG. 2. CCR5mim-Ig induces the CD4-bound state of the envelope glycoprotein. A-F) 293T cells transfected to express envelope glycoproteins of the indicated isolates were analyzed by flow cytometry with CCR5mim-Ig (left panels) or with CD4mim fused to the mouse IgG2a Fc region (CD4mim-mIg, right panels) in the presence of CCR5mim-Ig or nCSaR-Ig (a tyrosine-sulfated control peptide), as indicated. Specific enhancement of CD4mim-mIg binding in the presence of CCR5mim-Ig indicates induction or stabilization of the CD4-bound state of the envelope glycoprotein. G-H) Experiments similar to panels A-F, except that T20 fused to mouse IgG2a Fc region (T20-mIg) was used in the place of CD4mim-mIg. Specific enhancement of T20-mIg binding in the presence of CCR5mim-Ig indicates exposure of helical region 1 of gp41.

FIG. 3. CCR5 mimetic peptide recognition of clade B, C, and D isolates. 1.0 μg/mL of CD4-Ig, 2.5 μg/mL CCR5mim-Ig, or 3.0 μl/mL of HIV-1 patient sera was used to stain 293T cells transfected to express the envelope glycoprotein of the indicated isolates. Cells were analyzed by flow cytometry, and normalized to CD4-Ig binding. Viral clade is indicated in parentheses.

FIG. 4. Efficient neutralization of HIV-1 by eCD4-Ig. A) CD4-Ig and three variants assayed in (B)-(D) are represented. CD4-d1d2 (blue), IgG1 Fc (gray), and the CCR5-mimetic peptide (red) are shown. Note that E3-Ig, the best performing of these constructs, is eCD4-Ig. B) Comparison of CD4-Ig, E1-Ig, E2-Ig and E3-Ig. The R5-isolate ADA and the R5X4-isolate 89.6, modified to express GFP, were incubated with the indicated concentrations of inhibitor proteins. Infection is measured by flow cytometry. Order of neutralization efficiency is: E3-Ig (eCD4-Ig, red)>E2-Ig (purple)>E1-Ig (gray)>CD4-Ig (blue). C) A protein gel showing 1 μg of inhibitor proteins used in D), together with indicated amounts of BSA as a standard. D) Assay similar to that in B) except that E3-Ig (eCD4-Ig, red) is compared with CD4-Ig (blue), and the R5-isolate YU2 and the X4-isolate SG3 are included. IC₅₀ differences between CD4-Ig and eCD4-Ig are between 3-fold (YU2: IC₅₀ of eCD4-Ig: 2 nM) and 50-fold (89.6; IC₅₀ of eCD4-Ig: 600 pM).

FIG. 5. Exemplary amino acid sequence and nucleotide sequences for Fc (SEQ ID NO:18 and SEQ ID NO:19, respectively) and soluble CD4 (SEQ ID NO:20 and SEQ ID NO:21, respectively).

FIG. 6. e3-CD4-Ig neutralizes HIV-1 isolates markedly more efficiently than CD4-Ig. HIV-1 expressing the envelope glycoproteins of the indicated isolate was incubated with GHOST-CCR5 or GHOST-CXCR4 cells in the presence of varying concentrations of CD4-Ig or eCD4-Ig. Infection was assayed as percentage of cells expressing green fluorescent protein, and reported as concentrations necessary for 50% and 90% inhibition (IC₅₀, and IC₉₀, respectively). The fold difference between CD4-Ig and e3-CD4-Ig is also indicated, with the range of fold shown in parenthesis. The rightmost column indicates the enhancement of infection by CD4-Ig. No enhancement was observed with e3-CD4-Ig.

FIG. 7. e4-CD4-Ig neutralizes HIV-1 isolates markedly more efficiently than CD4-Ig.

### DETAILED DESCRIPTION OF THE INVENTION

Soluble forms of the HIV-1 receptor CD4 have been studied as potential therapeutics for many years. Soluble CD4 (sCD4), and the more bioavailable fusion protein, CD4-Ig, neutralize more broadly than any neutralizing antibody, and, unlike antibodies, they can irreversibly inactivate the envelope glycoprotein trimer. Nonetheless, several problems with CD4-Ig limit its therapeutic use. First, affinity of CD4 for the HIV-1 envelope glycoprotein is lower than that of efficient neutralizing antibodies. Second, CD4-Ig, like sCD4, enhances HIV-1 infection at low concentrations, a serious difficulty as local concentrations vary in vivo. This enhancement occurs because CD4-Ig bound at low valency promotes association of the envelope glycoprotein with the cellular coreceptor, CCR5 or CXCR4. Third, low concentration enhancement permits sufficient residual viral replication to enable viral escape. In addition to these difficulties, there remains a practical problem of using a protein therapeutic like CD4-Ig in resource-poor settings.

The HIV-1 envelope glycoprotein is a trimer of heterodimers composed of a surface (gp120) and a transmembrane (gp41) protein, processed from a single precursor (gp160) (Earl et al., Proc. Natl. Acad. Sci. USA, 87:648 (1990)). The entry of HIV-1 into its target cells requires expression of the cellular receptor CD4 and of a coreceptor, principally the chemokine receptors CCR5 or CXCR4 (Alkhatib et al., Science, 272:1955 (1996); Choe et al., Cell, 85:1135 (1996); Dalgleish et al., Nature, 312:763 (1984); Deng et al., Nature, 381:661 (1996); Doranz et al., Cell, 85:1149 (1996); Dragic et al., Nature, 381:667 (1996); and Feng et al., Science, 272:872 (1996)). Virus association with the primary receptor, CD4, triggers conformational changes in the envelope glycoprotein, gp120, that allows high-affinity binding to a coreceptor (Trkola et al., Nature, 384:184 (1996) and Wu et al., Nature, 184:179 (1996)). Association with CD4 and coreceptor induces a global rearrangement of the transmembrane envelope glycoprotein gp41. Rearrangement of gp41 proceeds in stages in which its trimeric coiled-coil formed by helical region 1 (HR1) is successively exposed, and then occluded by HR2 to form a six-helix bundle (Doms and Moore, J. Cell Biol., 151:F9 (2000); Eckert and Kim, Annu. Rev. Biochem., 70:777 (2001); and Weissenhorn et al., Nature, 387:426 (1997)). Six-helix bundle formation promotes mixing of viral and cellular membranes, and ultimately the entry of the viral capsid into the target cell. HIV-1 variants that utilize CCR5 as a coreceptor, R5 isolates, mediate transmission and predominate throughout most of the asymptomatic phase of infection. In many cases, with the decline in immune function, variants emerge that utilize coreceptors in addition to CCR5, in particular CXCR4 (Bleul et al., Proc. Natl. Acad. Sci. USA, 94:1925 (1997) and Connor et al., J. Exp. Med., 185:621 (1997)). Some of these variants (R5X4 isolates) utilize both principal coreceptors.

The ectodomains of CCR5 and CXCR4 consist of an amino-terminus and three extracellular loops held in position by seven transmembrane helices (Berger et al., Annu. Rev. Immunol., 17:657 (1999)). Entry of R5 HIV-1 isolates depends largely on the amino-terminus and second extracellular loop of CCR5 (Atchison et al., Science, 274:1924 (1996); Doranz et al., J. Virol., 71:6305 (1997); Farzan et al., J. Biol. Chem., 272:6854 (1997); and Rucker et al., Cell, 87:437 (1996)). An acidic, tyrosine-rich sequence within the CCR5 amino-terminus (residues 10-18) is especially important for viral entry. The tyrosines of this region are post-translationally modified by the addition of sulfate (Farzan et al., Cell, 96:667 (1999)). All R5 and R5X4 isolates examined to date are sensitive to the loss of one or more of these CCR5 sulfates. This dependence on tyrosine sulfate extends to multiple HIV-1 clades, including A, C, and D. Tyrosine-sulfated peptides based on the CCR5 amino-terminus can reconstitute entry with a CCR5-variant lacking its amino-terminus, indicating that this peptide can promote gp120 interaction with this otherwise non-functional CCR5 (Farzan et al., J. Biol. Chem., 277:40397 (2002) and Melikyan et al., Retrovirology, 4:55 (2007)).

Several groups of antibodies neutralize HIV-1 infection by binding gp120 or gp41 (Poignard et al., Annu. Rev. Immunol., 19:253 (2001)). These include antibodies that recognize the gp120 V3 loop, the CD4-binding site, and the base of the gp41 ectodomain (Muster et al., J. Virol., 67:6642 (1993); Roben et al., J. Virol., 68:4821 (1994); and Zwick et al., J. Virol., 75:10892 (2001)). An additional group of neutralizing antibodies, whose association with gp120 is enhanced by CD4, has been defined (Thali et al., J. Virol., 67:3978 (1993)). The epitopes of these CD4-induced (CD4i) antibodies overlap the coreceptor-binding region of gp120 (Trkola et al., 1996 and Wu et al., 2006)). Fab and scFv forms of these CD4i antibodies are considerably more effective at neutralizing HIV-1, presumably due to the greater access of these smaller forms to D4-bound gp120 (Choe et al., Cell, 114:161 (2003) and Labrijn et al., J. Virol., 77:10557 (2003)).

Some anti-gp120 antibodies are tyrosine-sulfated in their heavy-chain CDR3 loop. The sulfated antibodies from these individuals bind gp120 more efficiently in the presence of CD4, and are specifically inhibited from binding gp120-CD4 complexes by sulfated peptides based on the CCR5 amino-terminus. Two of these antibodies, 412d and E51, are potently neutralizing, and efficiently neutralize R5 and R5X4 isolates. E51 also efficiently binds and neutralizes X4 isolates.

Tyrosine-sulfated peptides based on the amino-terminus of CCR5 specifically inhibit HIV-1 entry, but at prohibitively high (about 50 μM) concentrations (Cormier et al., Proc. Natl. Acad. Sci. USA, 97:5762 (2000); Cormier et al., J. Virol., 75:5541 (2001); Farzan et al., 2002); and Farzan et al., J. Biol. Chem., 275:33516 (2000)). Interaction of these peptides with gp120 is nonetheless highly specific, as several lines of evidence make clear. First, these peptides compete with CD4i antibodies, but not CD4-binding site antibodies or those recognizing the GPGR tip of the V3 loop. The inefficiency of sulfated peptides based directly on the CCR5 amino-terminus arises from several factors. Second, these peptides have hydrophobic residues that likely associate with the remainder of CCR5, but which may create artifactual conformations and interfere with peptide solubility. A sulfated peptide derived from the CD4i antibody E51 that is quite similar to the CCR5 amino-terminus, but which has greater solubility, and which binds gp120 much more efficiently than CCR5-based peptides (Dorfman et al., J. Biol. Chem., 281:28529 (2006)).

Various forms of soluble CD4 (sCD4) and CD4-Ig have been used in clinical trials (McDougal et al., Curr. Opin. Immunol., 3:552 (1991) and Pincus, Antiviral Res., 33:1 (1996)). These inhibitors have been tested at high doses, up to 20-25 mg/kg (Jacobson et al., Antimicrob. Agents Chemother., 48:423 (2004)). While the efficacy of these variants has not been sufficiently impressive to further develop these proteins as therapeutics, these studies have made clear that soluble forms of CD4 can be introduced into humans without adverse consequences. An initial difficulty was that sCD4 has a short serum half-life. This problem was solved by fusing the first two domains of CD4 to the immunoglobulin (IgG) Fc-domain. This fusion confers on CD4 the ability to interact with the neonatal Fc receptor, to rescue antibodies from lysosomal degradation of protein absorbed into cells (He et al., Nature, 455:542 (2008)). The Fc domain also confers antibody effector functions onto CD4-Ig, including the ability to mediate antibody-dependent cell-mediated cytotoxicity, to access mucosal compartments, and to transport across the placenta.

IC₅₀s of sCD4 and CD4-Ig in vitro are markedly worse than those of most efficient neutralizing antibodies. Depending on isolate these IC₅₀s can range from 10-100 nM. One reason for this is that the affinity of CD4 for the trimeric HIV-1 envelope glycoprotein is low, for the simple reason that high affinity is not necessary to mediate efficient fusion, and the virus tends to occlude its CD4-binding site under immune pressure. Moreover, the dimerization of CD4-Ig affords no special advantage: the geometry of the envelope glycoprotein trimer permits association of only one CD4 per CD4-Ig. Another key reason for the poor IC₅₀ is that CD4-Ig enhances HIV-1 infection at low concentrations (Sullivan et al., J. Virol., 72:6332 (1998) and Sullivan et al., J. Virol., 72:4694 (1998)), a serious difficulty as local concentrations vary in vivo. This enhancement occurs because CD4-Ig can promote association of the envelope glycoprotein with the cellular coreceptor, CCR5 or CXCR4. The moderate affinity of CD4-Ig for the envelope glycoprotein, and its tendency to enhance entry, both contribute to viral escape in vivo. Indeed by further lowering its affinity for CD4, a virus increases the concentration at which entry can be enhanced. Because of their large surface area of contact, antibodies must interact with a number of poorly conserved residues, providing the virus with a ready means of escape. For example, the potent and widely studied antibody IgGb12 can be evaded without loss of fitness by a single mutation in the CD4-binding site of gp120, a proline to leucine change at residue 369 (Parren et al., J. Virol., 72:10270 (1998)).

In contrast, the present invention provides molecules encoding a fusion polypeptide, e.g., eCD4-Ig molecules, that are small enough to be expressed via scAAV, and the encoded CCR5-mimetic component interacts with a small, highly conserved region of gp120. Moreover, the encoded fusion polypeptide retains two key advantages of CD4-Ig relative to neutralizing antibodies. First, it promotes the irreversible inactivation of the envelope glycoprotein, observed as shed gp120 in the supernatant. Second, the virus must preserve its CD4-binding site.

### Definitions

A “vector” refers to a macromolecule or association of macromolecules that comprises or associates with a polynucleotide, and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest), a coding sequence of interest in vaccine development (such as a polynucleotide expressing a protein, polypeptide or peptide suitable for eliciting an immune response in a mammal), and/or a selectable or detectable marker.

“AAV” is adeno-associated virus, and may be used to refer to the naturally occurring wild-type virus itself or derivatives thereof. The term covers all subtypes, serotypes and pseudotypes, and both naturally occurring and recombinant forms, except where required otherwise. As used herein, the term “serotype” refers to an AAV which is identified by and distinguished from other AAVs based on capsid protein reactivity with defined antisera, e.g., serotypes including AAV-1 to AAV-8. For example, serotype AAV-2 is used to refer to an AAV which contains capsid proteins encoded from the cap gene of AAV-2 and a genome containing 5′ and 3′ ITR sequences from the same AAV-2 serotype. Pseudotyped AAV refers to an AAV that contains capsid proteins from one serotype and a viral genome including 5′-3′ ITRs of a second serotype. Pseudotyped rAAV would be expected to have cell surface binding properties of the capsid serotype and genetic properties consistent with the TPS serotype. The abbreviation “rAAV” refers to recombinant adeno-associated virus, also referred to as a recombinant AAV vector (or “rAAV vector”).

“Transduction,” “transfection,” “transformation” or “transducing” as used herein, are terms referring to a process for the introduction of an exogenous polynucleotide, e.g., a transgene in rAAV vector, into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by heterologousization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

An “rAAV vector” as used herein refers to an AAV vector comprising a polynucleotide sequence not of AAV origin (i.e., a polynucleotide heterologous to AAV), typically a sequence of interest for the genetic transformation of a cell. The term rAAV vector encompasses both rAAV virus particles and rAAV vector plasmids.

An “AAV virus” or “AAV viral particle” refers to a viral particle composed of at least one AAV capsid protein (preferably by all of the capsid proteins of a wild-type AAV) and an encapsidated polynucleotide. If the particle comprises a heterologous polynucleotide (i.e., a polynucleotide other than a wild-type AAV genome such as a transgene to be delivered to a mammalian cell), it is typically referred to as “rAAV”.

An “infectious” virus or viral particle is one that comprises a polynucleotide component which it is capable of delivering into a cell for which the viral species is trophic. The term does not necessarily imply any replication capacity of the virus.

A “replication-competent” virus (e.g., a replication-competent AAV, sometimes abbreviated as “RCA”) refers to a phenotypically wild-type virus that is infectious, and is also capable of being replicated in an infected cell (i.e., in the presence of a helper virus or helper virus functions). In the case of AAV, replication competence generally requires the presence of functional AAV packaging genes. rAAV vectors may be replication-incompetent in mammalian cells (especially in human cells) by virtue of the lack of one or more AAV packaging genes. Such rAAV vectors lack any AAV packaging gene sequences in order to minimize the possibility that RCA are generated by recombination between AAV packaging genes and an incoming rAAV vector. rAAV vector preparations may contain few if any RCA (less than about 1 RCA per 10² rAAV particles, e.g., less than about 1 RCA per 10¹² rAAV particles).

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A “transcriptional regulatory sequence” refers to a genomic region that controls the transcription of a gene or coding sequence to which it is operably linked. Transcriptional regulatory sequences of use in the present invention generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present invention are provided below.

“Host cells,” “cell lines,” “cell cultures,” “packaging cell line” and other such terms denote higher eukaryotic cells, such as mammalian cells including human cells, useful in the present invention, e.g., to produce recombinant virus or recombinant fusion polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

An “expression vector” is a vector comprising a region which encodes a gene product of interest, and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphonylation, lipidation, or conjugation with a labeling component.

An “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Increasing enrichments of the embodiments of this invention are increasingly more preferred. Thus, for example, a 2-fold enrichment is preferred, 10-fold enrichment is more preferred, 100-fold enrichment is more preferred, 1000-fold enrichment is even more preferred.

### Vectors for Gene Delivery

Delivery vectors include, for example, viral vectors, liposomes and other lipid-containing complexes, and other macromolecular complexes capable of mediating delivery of a gene to a host cell. Vectors can also comprise other components or functionalities that further modulate gene delivery and/or gene expression, or that otherwise provide beneficial properties. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., WO 92/08796; and WO 94/28143). Such marker genes can provide an added measure of control that can be advantageous in gene therapy contexts. A large variety of such vectors are known in the art and are generally available.

Vectors within the scope of the invention include, but are not limited to, isolated nucleic acid, e.g., plasmid-based vectors which may be extrachromosomally maintained, and viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Exemplary gene viral vectors are described below. Vectors may be administered via any route including, but not limited to, intramuscular, buccal, rectal, intravenous or intracoronary administration, and transfer to cells may be enhanced using electroporation and/or iontophoresis.

Retroviral Vectors

Retroviral vectors exhibit several distinctive features including their ability to stably and precisely integrate into the host genome providing long-term transgene expression. These vectors can be manipulated ex vivo to eliminate infectious gene particles to minimize the risk of systemic infection and patient-to-patient transmission. Pseudotyped retroviral vectors can alter host cell tropism.

Lentiviruses

Lentiviruses are derived from a family of retroviruses that include human immunodeficiency virus and feline immunodeficiency virus. However, unlike retroviruses that only infect dividing cells, lentiviruses can infect both dividing and nondividing cells. For instance, lentiviral vectors based on human immunodeficiency virus genome are capable of efficient transduction of cardiac myocytes in vivo. Although lentiviruses have specific tropisms, pseudotyping the viral envelope with vesicular stomatitis virus yields virus with a broader range (Schnepp et al., Meth. Mol. Med., 69:427 (2002)).

Adenoviral Vectors

Adenoviral vectors may be rendered replication-incompetent by deleting the early (E1A and E1B) genes responsible for viral gene expression from the genome and are stably maintained into the host cells in an extrachromosomal form. These vectors have the ability to transfect both replicating and nonreplicating cells and, in particular, these vectors have been shown to efficiently infect cardiac myocytes in vivo, e.g., after direction injection or perfusion. Adenoviral vectors have been shown to result in transient expression of therapeutic genes in vivo, peaking at 7 days and lasting approximately 4 weeks. The duration of transgene expression may be improved in systems utilizing cardiac specific promoters. In addition, adenoviral vectors can be produced at very high titers, allowing efficient gene transfer with small volumes of virus.

Adeno-Associated Virus Vectors

Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to infect replicating and nonreplicating cells and are believed to be nonpathogenic to humans.

Herpesvirus/Amplicon

Herpes simplex virus 1 (HSV-1) has a number of important characteristics that make it an important gene delivery vector in vivo. There are two types of HSV-1-based vectors: 1) those produced by inserting the exogenous genes into a backbone virus genome, and 2) HSV amplicon virions that are produced by inserting the exogenous gene into an amplicon plasmid that is subsequently replicated and then packaged into virion particles. HSV-1 can infect a wide variety of cells, both dividing and nondividing, but has obviously strong tropism towards nerve cells. It has a very large genome size and can accommodate very large transgenes (>35 kb).

### Plasmid DNA Vectors

Plasmid DNA is often referred to as “naked DNA” to indicate the absence of a more elaborate packaging system. Direct injection of plasmid DNA to myocardial cells in vivo has been accomplished. Plasmid-based vectors are relatively nonimmunogenic and nonpathogenic, with the potential to stably integrate in the cellular genome, resulting in long-term gene expression in postmitotic cells in vivo. Furthermore, plasmid DNA is rapidly degraded in the blood stream; therefore, the chance of transgene expression in distant organ systems is negligible. Plasmid DNA may be delivered to cells as part of a macromolecular complex, e.g., a liposome or DNA-protein complex, and delivery may be enhanced using techniques including electroporation.

### Amino Acid Sequence Modifications

It is envisioned that the sequence of any of the CCR5 mimetic peptide, CD4 polypeptide or Fc binding region of an Ig can be varied relative to a wild-type or parent sequence by amino acid substitution. Conservative amino acid substitutions are preferred, that is, for example, aspartic-glutamic as polar acidic amino acids; lysine/arginine/histidine as polar basic amino acids; leucine/isoleucine/methionine/valine/alanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide.

Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic; trp, tyr, phe.

The invention also envisions polypeptides with non-conservative substitutions. Non-conservative substitutions entail exchanging a member of one of the classes described above for another.

### Dosages and Dosage Forms

The amount of vector(s) or fusion polypeptide administered to achieve a particular outcome will vary depending on various factors including, but not limited to, the gene and promoter chosen, the condition, patient specific parameters, e.g., height, weight and age, and whether prevention or treatment is to be achieved. The vector or fusion polypeptide may be amenable to chronic use.

Vectors or fusion polypeptides of the invention may conveniently be provided in the form of formulations suitable for administration, e.g., into the blood stream (e.g., in an intracoronary artery). A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. Vectors or fusion polypeptides of the present invention may be formulated in solution at neutral pH, for example, about pH 6.5 to about pH 8.5, more preferably from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, more preferably from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions. If desired, solutions of the above compositions can also be prepared to enhance shelf life and stability. Therapeutically useful compositions of the invention can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The vectors or fusion polypeptides can be provided in a dosage form containing an amount of a vector effective in one or multiple doses. For viral vectors, the effective dose may be in the range of at least about 10⁷ viral particles, e.g., about 10⁹ viral particles, 10¹¹ viral particles or 10¹⁴ viral. As noted, the exact dose to be administered is determined by the attending clinician, but is may be in 1 mL phosphate buffered saline. For delivery of plasmid DNA alone, or plasmid DNA in a complex with other macromolecules, the amount of DNA to be administered will be an amount which results in a beneficial effect to the recipient. For example, from 0.0001 to 1 mg or more, e.g., up to 1 g, in individual or divided doses, e.g., from 0.001 to 0.5 mg, or 0.01 to 0.1 mg, of DNA can be administered. For delivery of the fusion polypeptide, the amount administered is an amount which results in a beneficial effect to the recipient. For example, from 0.0001 to 100 g or more, e.g., up to 1 g, in individual or divided doses, e.g., from 0.001 to 0.5 g, or 0.01 to 0.1 g, of fusion polypeptide can be administered.

Administration of the vector or fusion polypeptide in accordance with the present invention may be continuous or intermittent, depending, for example, upon the recipient's physiological condition, whether the purpose of the administration is therapeutic or prophylactic, and other factors known to skilled practitioners. The administration of the vector or fusion polypeptide may be essentially continuous over a preselected period of time or may be in a series of spaced doses. Both local and systemic administration is contemplated.

One or more suitable unit dosage forms comprising the vector or fusion polypeptide, which may optionally be formulated for sustained release, can be administered by a variety of routes including oral, or parenteral, including by rectal, buccal, vaginal and sublingual, transdermal, subcutaneous, intravenous, intramuscular, intraperitoneal, intrathoracic, intrapulmonary and intranasal routes. The formulations may, where appropriate, be conveniently presented in discrete unit dosage forms and may be prepared by any of the methods well known to pharmacy. Such methods may include the step of bringing into association the vector or fusion polypeptide with liquid carriers, solid matrices, semi-solid carriers, finely divided solid carriers or combinations thereof, and then, if necessary, introducing or shaping the product into the desired delivery system.

Pharmaceutical formulations containing the vector or fusion polypeptide can be prepared by procedures known in the art using well known and readily available ingredients. For example, the agent can be formulated with common excipients, diluents, or carriers, and formed into tablets, capsules, suspensions, powders, and the like. The vectors or fusion polypeptides of the invention can also be formulated as elixirs or solutions for convenient oral administration or as solutions appropriate for parenteral administration, for instance by intramuscular, subcutaneous or intravenous routes.

The pharmaceutical formulations of the vectors or fusion polypeptides can also take the form of an aqueous or anhydrous solution or dispersion, or alternatively the form of an emulsion or suspension.

Thus, the vector or fusion polypeptide may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

For administration to the upper (nasal) or lower respiratory tract by inhalation, the vector or fusion polypeptide is conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the vector or fusion polypeptide may be administered via nose drops, a liquid spray, such as via a plastic bottle atomizer or metered-dose inhaler. Typical of atomizers are the Mistometer (Wintrop) and the Medihaler (Riker).

For topical administration, the vectors or fusion polypeptides may be formulated as is known in the art for direct application to a target area. Conventional forms for this purpose include wound dressings, coated bandages or other polymer coverings, ointments, creams, lotions, pastes, jellies, sprays, and aerosols, as well as in toothpaste and mouthwash, or by other suitable forms. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredients can also be delivered via iontophoresis, e.g., as disclosed in U.S. Pat. Nos. 4,140,122; 4,383,529; or 4,051,842. The percent by weight of a therapeutic agent of the invention present in a topical formulation will depend on various factors, but generally will be from 0.01% to 95% of the total weight of the formulation, and typically 0.1-25% by weight.

When desired, the above-described formulations can be adapted to give sustained release of the active ingredient employed, e.g., by combination with certain hydrophilic polymer matrices, e.g., comprising natural gels, synthetic polymer gels or mixtures thereof.

Drops, such as eye drops or nose drops, may be formulated with an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The vector or fusion polypeptide may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; mouthwashes comprising the composition of the present invention in a suitable liquid carrier; and pastes and gels, e.g., toothpastes or gels, comprising the composition of the invention.

The formulations and compositions described herein may also contain other ingredients such as antiviral agent, antimicrobial agents or preservatives.

### AAV Delivery

In particular, for delivery of an AAV vector of the invention to a tissue such as muscle, any physical or biological method that will introduce the vector into the muscle tissue of a host animal can be employed. Vector means both a bare recombinant vector and vector DNA packaged into viral coat proteins, as is well known for AAV administration. Simply dissolving an AAV vector in phosphate buffered saline has been demonstrated to be sufficient to provide a vehicle useful for muscle tissue expression, and there are no known restrictions on the carriers or other components that can be coadministered with the vector (although compositions that degrade DNA should be avoided in the normal manner with vectors). Pharmaceutical compositions can be prepared as injectable formulations or as topical formulations to be delivered to the muscles by transdermal transport. Numerous formulations for both intramuscular injection and transdermal transport have been previously developed and can be used in the practice of the invention. The vectors can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of the AAV vector as a free acid (DNA contains acidic phosphate groups) or a pharmacologically acceptable salt can be prepared in water suitably mixed with a surfactant such as hydroxypropylcellulo se. A dispersion of AAV viral particles can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating the AAV vector in the required amount in the appropriate solvent with various of the other ingredients enumerated above, as required, followed by filtered sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from the previously sterile-filtered solution thereof.

For purposes of topical administration, dilute sterile, aqueous solutions (usually in about 0.1% to 5% concentration), otherwise similar to the above parenteral solutions, are prepared in containers suitable for incorporation into a transdermal patch, and can include known carriers, such as pharmaceutical grade dimethylsulfoxide (DMSO).

The therapeutic compounds of this invention may be administered to a mammal alone or in combination with pharmaceutically acceptable carriers. As noted above, the relative proportions of active ingredient and carrier are determined by the solubility and chemical nature of the compound, chosen route of administration and standard pharmaceutical practice.

The dosage of the present therapeutic agents which will be most suitable for prophylaxis or treatment will vary with the form of administration, the particular compound chosen and the physiological characteristics of the particular patient under treatment. Generally, small dosages will be used initially and, if necessary, will be increased by small increments until the optimum effect under the circumstances is reached.

The invention will be described by the following nonlimiting examples.

### EXAMPLE 1

Sulfated peptides based on the N-terminus of CCR5 specifically block HIV-1 infection, but only at 50-100 μM concentrations (Cormier et al., 2000 and Farzan et al., J. Exp. Med., 193:1059 (2001)). The ability of pΔE51-Ig, but not pR5-Ig, to precipitate gp120 in the presence and absence of CD4 suggested that pΔE51-Ig may be more effective at inhibiting HIV-1 infection. CD4-Ig and pΔE51-Ig were compared for their ability to inhibit entry of an infectious HIV-1 variant expressing GFP and the envelope glycoprotein of the R5X4 isolate 89.6. Virus was incubated with the CD4-positive T-cell line PM1 and the indicated peptide-fusion proteins for one hour, and then washed. Consistent with its ability to bind gp120, pΔE51-Ig inhibited infection markedly, with an IC₅₀ reproducibly observed between 1 and 2 μM. Expectedly, CD4-Ig blocked infection in the nanomolar range. Similar results were obtained using a broader range of envelope glycoproteins of clade B isolates. Pseudovirus infection mediated by the envelope glycoproteins of the R5 isolates ADA, YU2, JR-FL, or of the X4 isolates HXB2 or NL4-3 was inhibited by pΔE51-Ig comparably to that of the 89.6 isolate. A control HIV-1 pseudotyped with the vesicular stomatitis virus G protein was not inhibited by pΔE51. These data show that pΔE51-Ig neutralizes clade B HIV-1 isolates much more efficiently than does pR5-Ig, a peptide-Fc fusion bearing residues identical to the CCR5-amino terminus.

The elimination of additional residues separating tyrosine 3 and tyrosine 10 of CCR5 may permit binding of an additional sulfated tyrosine to gp120 without the entropic penalty of folding the small intervening loop. These CCR5 sulfotyrosines are important for HIV-1 entry (Cormier et al., 2000; Farzan et al., 1998; and Farzan et al., 1999). Note also that the final tyrosine of pΔE51, which does not align with CCR5 tyrosines, contributes less to gp120 association than the other four tyrosines.

Finally, the first glycine and the last two residues of pΔE51-Ig do not contribute to binding or neutralization (Dorfman et al., 2006). Therefore, one CCR5-mimetic peptide is 12 amino-acids: DY*ADY*DGGY*Y*Y*D (“CCRmim”; Y* indicates sulfotyrosine). Its fusion with the Fc domain of human IgG1 is “CCR5mim-Ig”.

### EXAMPLE 2

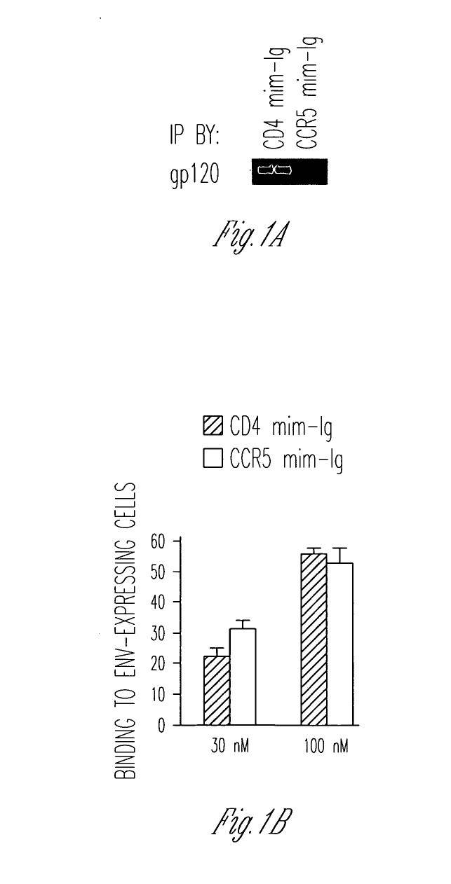

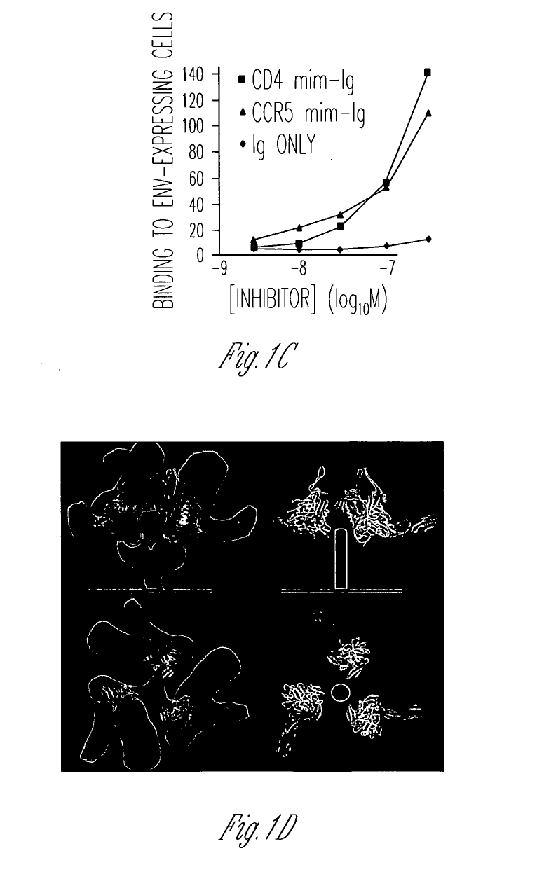

FIG. 1 uses several peptides to further explore the effect of CCR5mim-Ig on the envelope glycoprotein. Note that to perform the flow cytometry experiments, forms of the envelope glycoproteins were used that were truncated in their cytoplasmic domains, a modification that greatly enhances their cell-surface expression. In addition to CCR5mim-Ig, a 27-amino-acid CD4-mimetic peptide (“CD4mim”), a phage-improved version of a peptide originally described by Carlo Vita's laboratory (Martin et al., Nat. Biotechnol., 21:71 (2003)) was used. This peptide has been crystallized with gp120, and was shown to induce a gp120 conformation identical to CD4-bound gp120 (Huang et al., Structure, 13:755 (2005)). Its Fc-fusion protein (“CD4mim-Ig”) binds cell-expressed envelope glycoprotein with an avidity comparable to that of CCR5mim-Ig (FIG. 1B). However, it binds soluble monomeric gp120 much better than CCR5mim-Ig (FIG. 1A). Interestingly, its binding curve to cell-expressed envelope glycoprotein reproducibly crosses that of CCR5mim-Ig (FIG. 1C).

These data suggest that, at low concentrations, the high avidity CCR5mim-Ig binds more efficiently than CD4mim-Ig. However, unlike CCR5mim-Ig, multiple CD4mim-Ig molecules bind the envelope glycoprotein at higher concentrations. The geometry of the envelope glycoprotein trimer, determined by cryo-electron microscopy (Liu et al., 2008), is consistent with this observation. The C-terminus of domain 2 of CD4 is positioned far from the trimer axis at a distance that would prevent CD4-Ig from binding more than one monomer, and, relevant to CD4mim as well, requires a very long connecting sequence to travel around the bulky gp120 molecule. To examine the location of the sulfate-binding pockets of gp120 in the trimer, the gp120 from the gp120/CD4/412d complex was fit into the gp120/CD4/17b complex previously fit to the cryoEM density. CD4i antibodies 17b and 412d bind gp120 in the same orientation. The sulfate-binding pockets of gp120 are positioned in this model at the top of the trimer stalk, close to the trimer axis, permitting both peptides from CCR5mim-Ig to associate the envelope glycoprotein. Collectively the data of FIG. 1 suggest that a single CCR5mim-Ig binds two gp120 monomers of the envelope glycoprotein, whereas CD4mim-Ig binds only one.

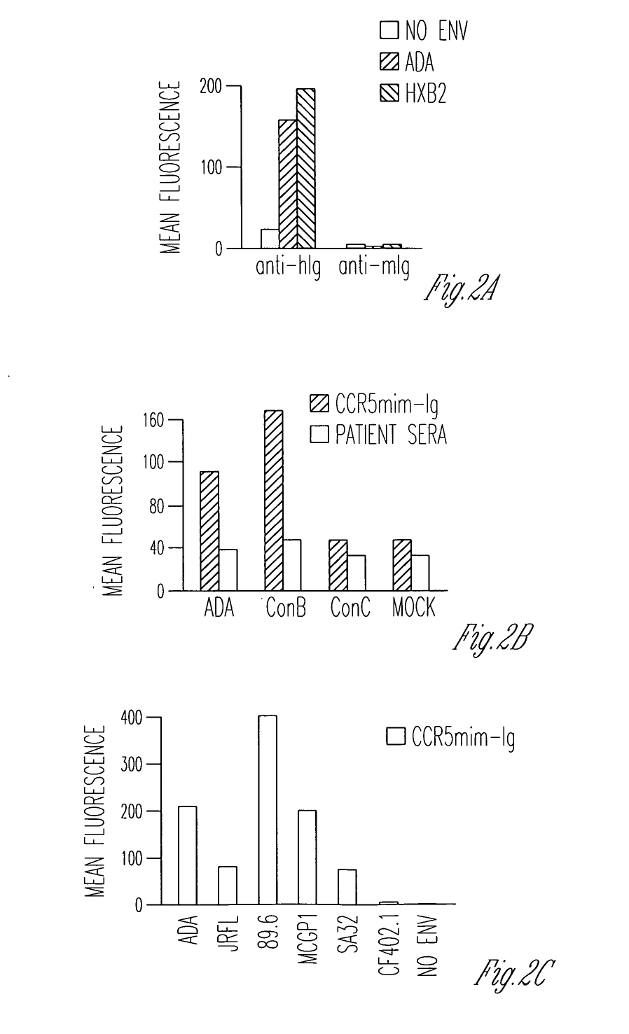

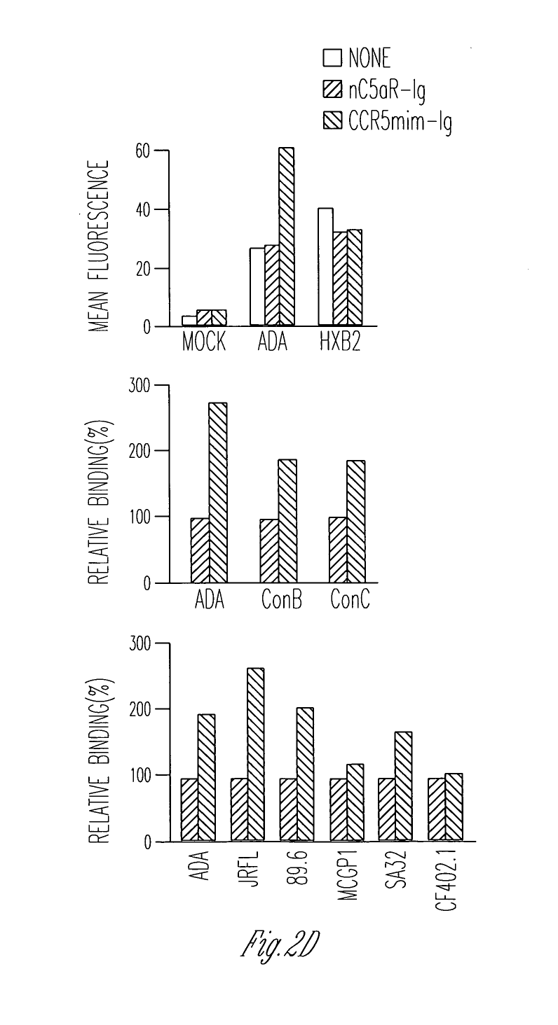

A series of studies of CCR5mim-Ig and CD4mim-Ig was conducted using a wider range of isolates. To do so, the human Fc of CD4mim-Ig was replaced with that of mouse Ig2a. Use of murine Fc is indicated as “-mIg”, thus CD4mim-mIg, which allows a comparison of binding of both fusion proteins in the same experiment. For example, in FIG. 2, left top panel, an anti-human Fc secondary antibody, but not an anti-mouse Fc secondary antibody, recognizes CCR5mim-Ig. It is well established that CD4 binding can induce or stabilize the CCR5-binding site of the envelope glycoprotein. It was hypothesized that the reverse is true, namely, that CCR5mim-Ig can by itself induce the CD4-bound conformation of the envelope glycoprotein. Therefore, the effect of CCR5mim-Ig on the ability of CD4mim-mIg to bind cell-surface expressed envelope glycoprotein was examined. A tyrosine-sulfated control for CCR5mim-Ig, based on the amino-terminus of the C5a receptor, was also used. This construct, nC5aR-Ig, like CCR5mim-Ig, is fused to a human Fc domain. As FIG. 2 shows, CCR5mim-Ig, but not nC5aR-Ig, enhanced the association of CD4mim-mIg with the envelope glycoprotein of several clade B (ADA, ConB, 89.6) and clade C isolates (SA32, ConC). This suggests that the CCR5-mimetic peptide can induce or stabilize the CD4-bound conformation of the envelope glycoprotein.

In addition to promoting CCR5 association, CD4 also induces exposure of helical region 1 (HR1) of gp41; peptides based on helical region 2 (including Enfuvirtide, a peptide in current clinical use (Jiang et al., BBRC, 269:641 (2000)) that recognize HR1 (Jiang et al., Nature, 365:113 (1999)) bind envelope glycoprotein better in the presence of sCD4 (Si et al., PNAS USA, 101:5036 (2004)). The CCR5-mimetic peptide induces a CD4-bound conformation in the envelope glycoprotein, it too would enhance binding of HR2-like peptides. Accordingly, one such peptide, T20, was fused to the Fc-region of murine IgG2a (“T20-mIg”) and it was determined whether CCR5mim-Ig could enhance its association to HR1. As shown in bottom panel of FIG. 2, CCR5mim-Ig by itself robustly enhanced T20-Ig association with all three clade B isolates assayed. Thus, like soluble CD4, CCR5mim-Ig can expose HR1 of gp41. Collectively, the data in FIG. 2 indicate that CCR5mim-Ig by itself can induced conformation changes in the envelope glycoprotein similar to those induced by CD4.

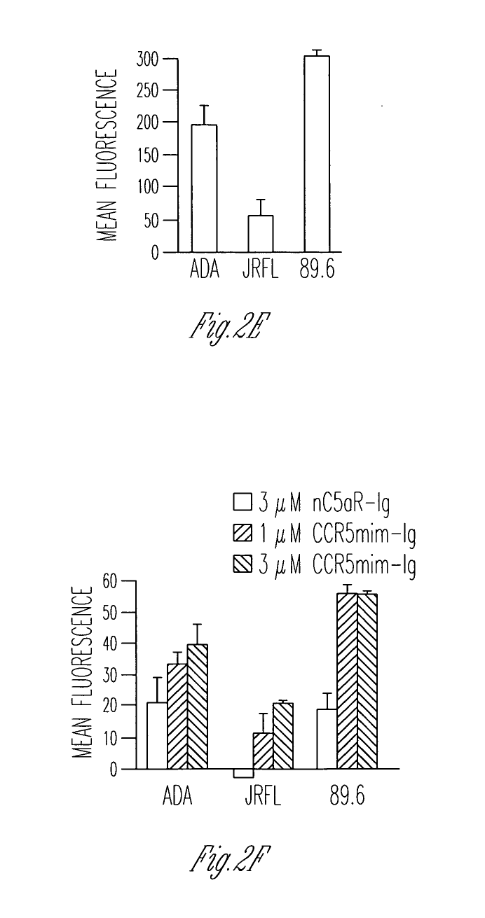

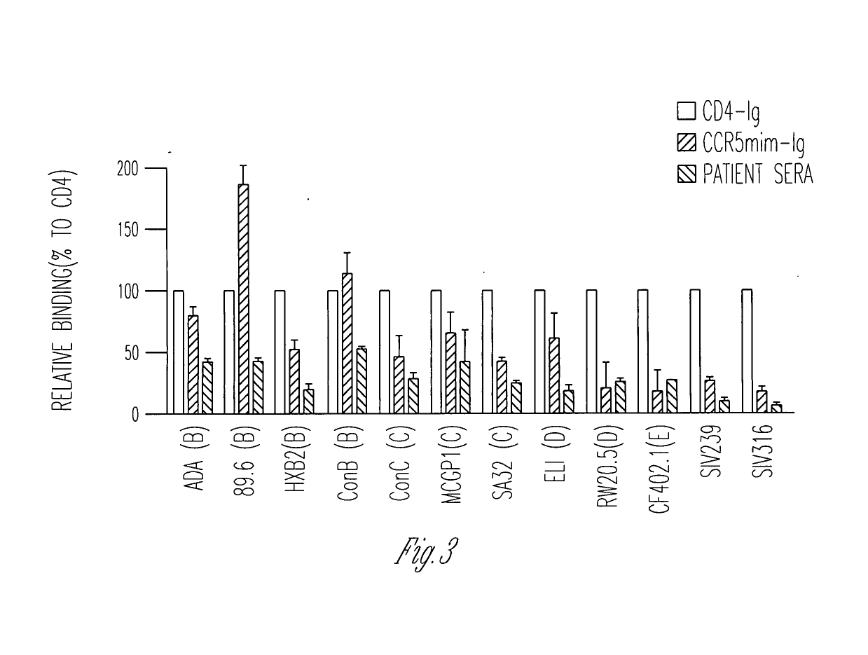

The ability of CCR5mim-Ig to bind the envelope glycoproteins of a broader range of HIV-1 isolates as well two related SIV isolates was examined (FIG. 3). To roughly indicate cell-surface expression of these various envelope glycoproteins, parallel flow cytometry assays were conducted with CD4-Ig, or with a mixture of sera from patients infected with clade B isolates. CCR5mim-Ig efficiently bound all four clade B, and surprisingly, all three clade C envelope glycoproteins. It also efficiently bound one of two clade D envelope glycoproteins. It poorly bound the envelope glycoprotein of the single clade E isolate tested, or those of SIV, despite the strong dependence of SIV on CCR5's sulfotyrosines. Thus, CCR5mim-Ig appears to recognize most clade B and clade C isolates. Subtle differences between the CCR5 amino terminus and CCR5mim likely account for the inability of SIV envelope glycoproteins in particular to associate with CCR5mim-Ig.

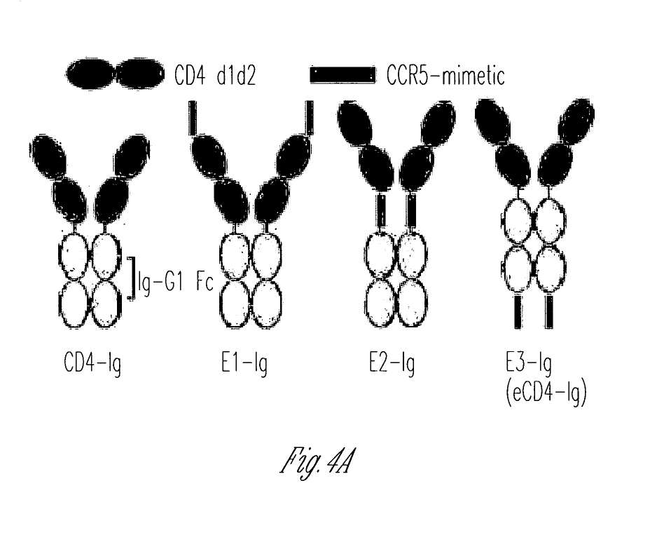

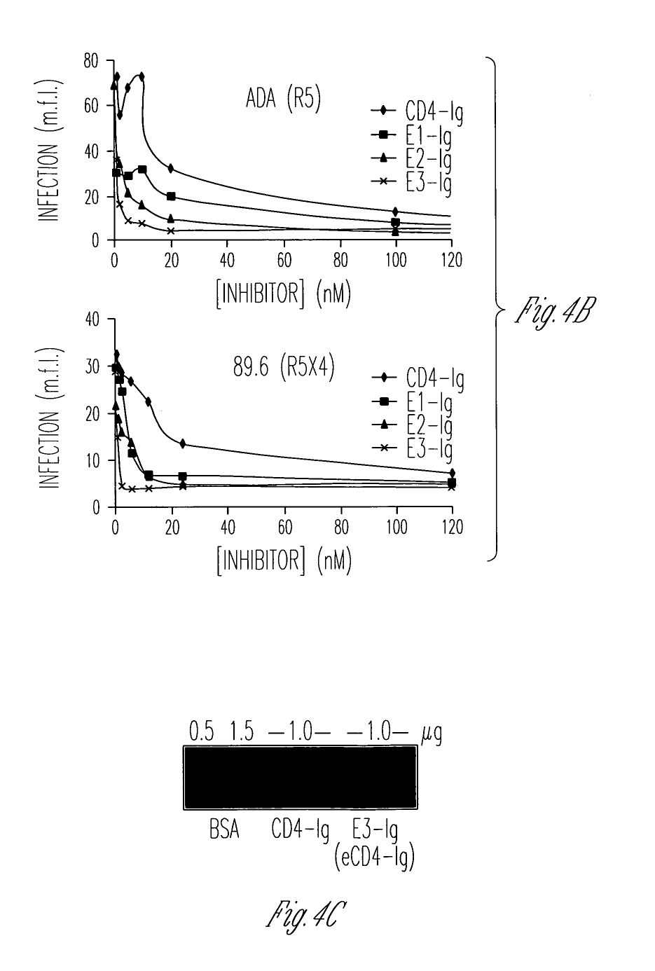

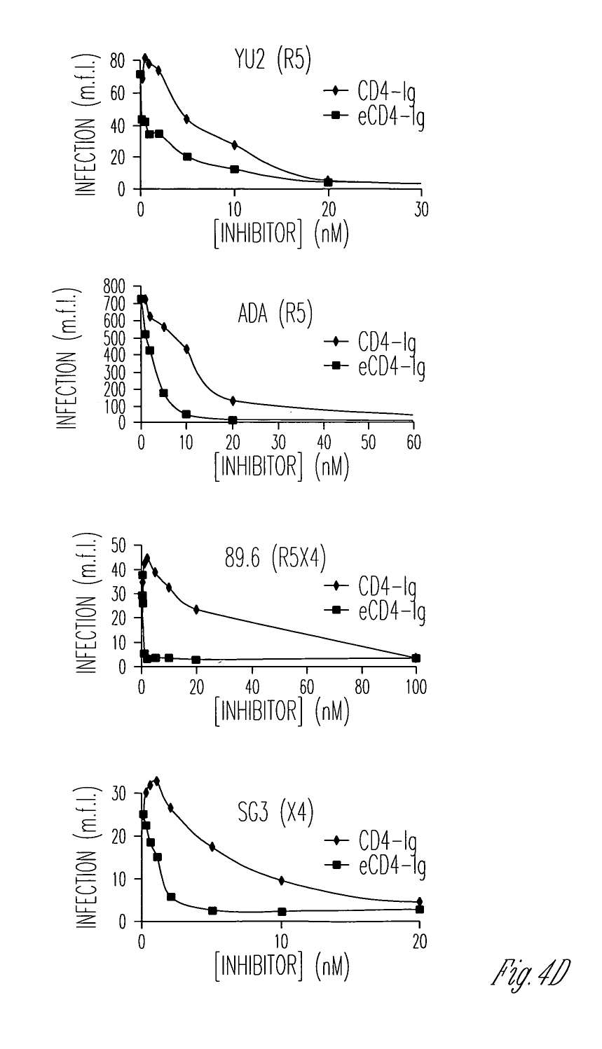

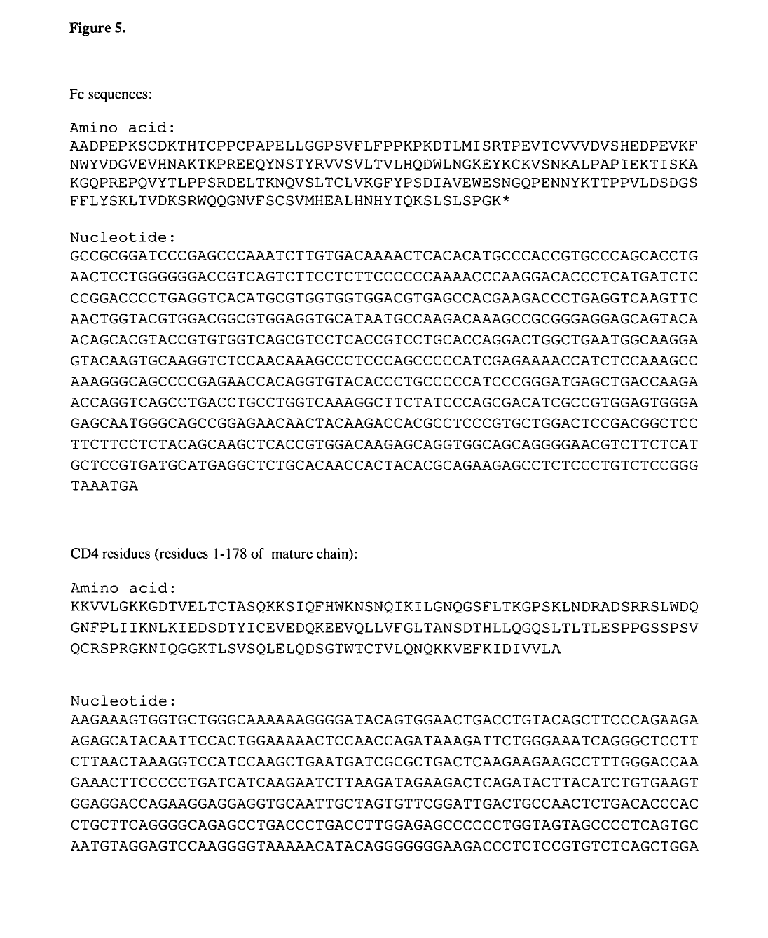

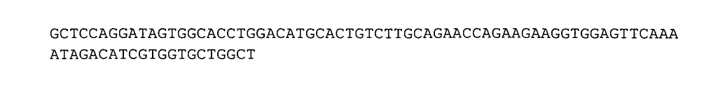

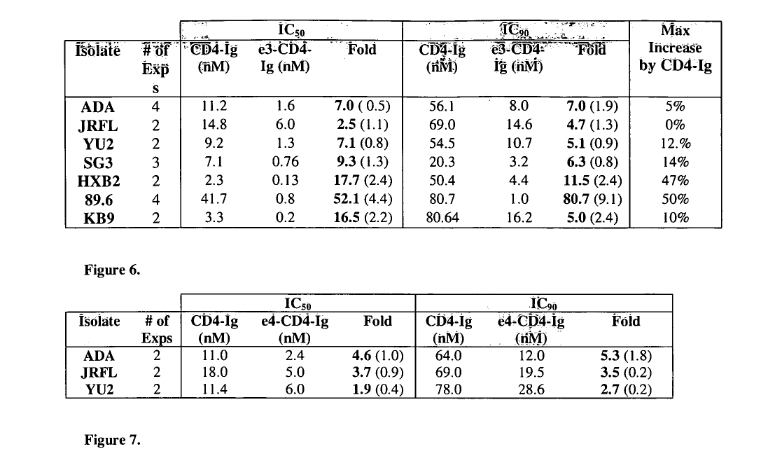

Next, the ability of three CD4-Ig/CCR5mim fusion constructs and unmodified CD4-Ig (represented in FIG. 4A) to neutralize infection by ADA and 89.6 isolates were compared (FIG. 4B). The CCR5-mimetic peptide was placed in three locations of CD4-Ig, at the amino-terminus (E1-Ig), in the linker region between CD4-d1d2 and the Fc region (E2-Ig), or at the carboxy-terminus (E3-Ig, renamed eCD4-Ig). The CCR5 mimetic peptide was connected by a four amino-acid linker (GSGG). Consistently, all three fusion constructs outperformed unmodified CD4-Ig, with eCD4-Ig (E3-Ig) outperforming the other constructs. In additional studies, eCD4-Ig neutralized two R5 isolates, one R5X4 isolate and one X4 isolate markedly more efficiently than CD4-Ig. Part of the reason for eCD4-Ig's more efficient neutralization is visible at concentrations below 5 nM: CD4-Ig increases infection, but eCD4-Ig clearly decreases infection. Thus, eCD4-Ig more effectively neutralizes HIV-1 in part because it does not enhance infection at low concentrations.

### EXAMPLE 3

AAV is a single-stranded DNA parvovirus that does not cause human disease. It can be engineered to deliver a single gene-of-interest, without co-expression of viral proteins. The viral-vector particle is expressed from cells transfected with three plasmids: one that encodes the viral rep and cap proteins, one containing the adenovirus E2A and E4 genes, and one expressing a transgene-of-interest bounded by AAV inverted terminal repeats (ITRs), which facilitate its nuclear replication. Relying on the host-cell polymerase, AAV replicates efficiently in post-mitotic cells including those of muscle. Lacking necessary viral genes, AAV vectors do not integrate, but express as an episomal concatamer of viral genomes. The rate-limiting step in transgene expression is the formation of the second, complementary strand of the viral genome. This bottleneck can be circumvented by encoding the complement of the gene in the viral genome, a so-called self-complementary (sc) AAV. scAAV improves sustained transgene expression by 20-fold or more. For example, a recent study of the prophylactic use of AAV-delivered protein reported 10-15 μg/mL inhibitor concentrations one year later with a conventional AAV vector, whereas the scAAV vector produced protein at greater than 250 μg/mL one year out, and more than 150 μg/mlL two years out (Johnson et al., Nat. Med., 15:901 (2009)). However, use of scAAV imposes a size limitation on the transgene, to approximately 1.5 kB or 500 amino acids for the expressed protein, which permits expression of single-chain/Fc fusions (scFv-Ig), as well as CD4-Ig and eCD4-Ig.

AAV vectors have enjoyed some major clinical successes, usually by complementing a missing protein in a monogenic disease, and more than 40 protocols for human trials have been FDA-approved. Animal studies also have been encouraging. For example, in a study by Phil Johnson and colleagues, 2 of 3 rhesus macaques expressing CD4-Ig, and 4 of 6 expressing scFv-Ig were protected from a high-dose intravenous challenge with SIV_{mac}316 (Johnson et al., 2009). Those that were not protected raised anti-inhibitor antibodies, but even in these cases 3 of 3 animals survived an SIV_{mac}316 challenge, whereas 4 of 6 control animals died.

Although scAAV vectors have largely solved the problem of low-risk, long-term expression of a protein therapeutic and can be delivered through a single injection that results in sustained titers necessary to control virus for more than two years, suppressing an established HIV-1 infection is far more challenging than preventing transmission. In an ongoing infection, the scale of viral replication and diversity is far greater, and there are numerous local environments from which resistant variants can emerge. To succeed, one must use an inhibitor that necessarily extracts a high fitness cost from an escaping virus. Antibodies typically recognize a large gp120 surface area including variable residues that easily facilitate viral escape. In theory, CD4-Ig would be an ideal inhibitor because the virus must bind the cellular CD4 to infect cells. However CD4-Ig binds the envelope glycoprotein with only moderate affinity, and it enhances entry at low concentrations. By appending a small CCR5-mimetic peptide to CD4-Ig, the resulting molecule had markedly increased neutralization efficiency and prevented low-concentration enhancement, probably because the peptide prevents association of gp120 with coreceptor. Moreover, eCD4-Ig remains small enough for expression by scAAV. The CCR5-mimetic component recognizes a range of clade B and C isolates in a conserved region of gp120.

The rhesus macaque provides a well established animal model of HIV-1 infection. Although not infectable with HIV-1, macaques can be infected with similar SIV viruses that cause AIDS-like symptoms. A commonly studied SIV isolate, SIV_{mac}239, has been modified to express the HIV-1 envelope glycoprotein, and adapted in macaques to replicate efficiently and cause disease (Karlsson et al., J. Virol., 71:4218 (1997) and Reimann et al., J. Virol., 70:6922 (1996)). This simian/human immunodeficiency virus (SHIV) can be used to study vaccines and inhibitors designed to target the HIV-1 envelope glycoprotein. SHIV89.6P causes a more rapid progression to AIDS than does SIV_{mac}239 in macaques or HIV-1 in humans (Karlsson et al., 1997 and Reimann et al., 1996) and so serves as a model for late-stage disease. SHIV-89.6P tends to use CXCR4 in vivo (Veazey et al., J. Exp. Med., 198:1551 (2003)). For this reason, the less efficient R5 SHIV-SF162P3 (Harouse et al., J. Virol., 75:1960 (2001) and Pahar et al., Virology, 363:36 (2007)), is used in studies of AAV-mediated prophylaxis, because HIV-1 transmission is mediated by R5 isolates.

SHIV-89.6P was used to assess the therapeutic efficacy of eCD4-Ig for several reasons. First, SHIV-89.6P is highly sensitive to eCD4-Ig, but replicates robustly. Thus, it is more likely to prove the principal that a CCR5-mimetic peptide can enhance the function of CD4-Ig in vivo. Second, the most likely initial human recipients of scAAV-delivered eCD4-Ig are late-stage patients receiving salvage therapy, and SHIV 89.6P may better model late-stage infection than SHIV-SF162P or SIV_{mac}239 (Karlsson et al., 1997 and Reimann et al., 1996). Moreover these patients, like SHIV-89.6-infected macaques, are less likely to raise antibody responses to AAV-expressed transgenes. Finally, there is more detailed structural information about the HIV-1 envelope glycoprotein than SIV, and more functional studies of 89.6 than SF162 envelope glycoproteins (Farzan et al., 1999). This information allows for understanding the mechanisms by which viruses escape CD4-Ig and eCD4-Ig.

Immune clearance of scAAV-delivered scFv-Ig and CD4-Ig is a frequent and formidable problem in healthy, uninfected macaques, although perhaps less of a concern in late-stage infections. A second major problem is that of viral escape, a much greater concern with therapeutic applications of scAAV than with prophylactic ones. Thus, the studies evaluate the use scAAV-delivered protein inhibitors in infected individuals with a therapeutic with important advantages over neutralizing antibodies and unmodified CD4-Ig. Six weeks following infection with a variant of SIV with a HIV envelope glycoprotein, animals are inoculated with an scAAV vector expressing CD4-Ig (3 animals), e5-CD4-Ig (3 animals), or with vector alone (3 animals). CD4-Ig titers and viral loads are monitored on a weekly basis thereafter. The envelope glycoprotein genes of viruses isolated at four intervals are characterized for their ability to escape from CD4-Ig and e5-CD4-Ig, coreceptor usage, and sensitivity to antibody neutralization. Animals are also monitored for unexpected side effects an anti-inhibitor responses.

Two criteria define success: (1) a statistically significant difference in viral loads between animals expressing CD4-Ig and those expressing e5-CD4-Ig, (2) a 10-fold drop in viral loads in animals expressing e5-CD4-Ig—sustained for one year—relative to control animals.

Description of self-complementary AAV vectors. Although a major improvement in the efficiency of transgene expression, self-complementary AAV (scAAV) uses nearly identical vector constructs as single-stranded AAV, except that one inverted terminal repeat (ITR) is modified in its terminal resolution site (trs). This modification prevents the viral endonuclease from nicking the double-strand DNA at the trs, preventing strand separation. Instead both forward and reverse strands remain linked by the modified ITR. The result is that final packaged genome includes forward and reverse strands encoding the transgene—if it is small enough to be packaged by scAAV. The practical limit on scAAV transgene size is roughly 2.5 kb, but this needs to include exogenous promoters and other factors that enhance transgene expression (730 bp), and a polyadenylation site (270 bp). The size of an expressed protein is therefore limited to approximately 500 amino-acids.

Generation of scAAV-CD4-Ig and scAAV-eCD4-Ig vectors. Accordingly, a modified AAV-2-based commercial system (Stratagene) is employed to provide scAAV stock. This system employs three plasmids co-transfected into a 293T cell line made stable for two adenovirus helper proteins (E1A and E1B). One plasmid (pAAV-RC) expresses the rep and cap AAV genes, necessary for genome replication and assembly, respectively. A second (pHelper) provides additional adenovirus genes (E2A, E4) that promote AAV replication. The final plasmid encodes the gene-of-interest (CD4-Ig, and eCD4-Ig) bounded by the AAV ITRs, one of which contains a deletion in its trs, as described above. The transgene itself is driven by a CMV promoter, includes an intron between the promoter and leader sequence to improve expression, and a bovine growth hormone polyadenylation site. The transgene encodes the rhesus CD4 leader sequence, and its first two immunoglobulin-like domains fused to a linker and the Fc domain of rhesus IgG1. In the case of eCD4-Ig, a sequence encoding a four amino-acid linker (GSGG) and a twelve amino-acid CCR5-mimetic peptide (DYADYDGGYYYD; SEQ ID NO:15) are included. To ensure comparable packaging efficiency of the eCD4-Ig and CD4-Ig transgenes, the CD4-Ig transgene will be generated by introducing a stop codon into the eCD4-Ig transgene, thus ensuring equal length of the two packaged transgene constructs.

Administration of scAAV vectors. scAAV vector particles expressing CD4-Ig, eCD4-Ig, and control are harvested from transfected cells, concentrated by centrifugation as previously described in Lewis et al., J. Virol., 76:8769 (2002), and titers quantified using bead capture of the AAV virions and a fluorescent quantitation of the viral genome (Cell Biolabs). 2×10¹³ vector genomes are administered via four injections, two per quadriceps, to 16 male Indian origin rhesus macaques (6 receiving scAAV-CD4-Ig, 6 receiving scAAVeCD4-Ig and 4 control animals receiving an scAAV vector lacking an expressible transgene).

Measurement of CD4-Ig and eCD4-Ig titers and functional activity. CD4-Ig and eCD4-Ig expression in animals are measured using an ELISA system with immobilized murine anti-rhesus CD4 antibody. A complementary ELISAassay is also employed in which a form of the SIV_{mac}239 gp120 lacking its sulfate-binding pockets as a precaution (altering residues 419-421, see FIG. 4C for the HIV-1 gp120 equivalent) is immobilized. Note that this second ELISA formats cannot be applied after SHIV challenge due to cross-reactivity in anti-sera recognizing HIV-1 and SIV envelope glycoproteins. In parallel, the neutralization efficiency of serial dilutions of sera from infected animals are assayed by measuring inhibition of HIV-1 (SF162 and 89.6 isolates), as previously described (Choe et al., 2003 and Johnson et al., 2009).

Measurement of anti-CD4-Ig and -eCD4-Ig antibodies. The presence of anti-inhibitor antibodies are assayed as previously described (Johnson et al., 2009). Sera from scAAV inoculated macaques are incubated at 1:100 dilution with immobilized soluble rhesus CD4, CD4-Ig, eCD4-Ig, CCR5mim-Ig, or Ig-only (Ig indicating the rhesus IgG1 Fc), and measured in an ELISA format as above. If anti-inhibitor antibodies are detected, variants of each construct are made to determine the major target epitopes. For example, if CD4-Ig, but not soluble CD4 or Ig-only, is recognized, the linker between these domains is modified and reassayed.

Monitoring for side effects of transgene expression. Adverse reactions to recombinant antibody therapeutic agents typically consist of mild, flu-like symptoms that decrease in severity with subsequent dosing. Veterinary staff monitor animals for suggestive clinical signs such as anorexia and lethargy as well as any local inflammatory reaction at the intramuscular injection site. Following low-dose viral challenge and euthanasia, complete necropsies are performed and tissues examined by veterinary pathologists for signs of toxicity. Inflammation at the injection site is characterized via immunohistochemistry to determine the degree of cellular immune response, if any, to AAV infected myocytes. Histopathology of renal glomeruli is performed to assess potential immune-complex deposition. Immunohistochemical staining for CD4 allow specific detection of the CD4-Ig and eCD4-Ig in glomerular mesangium or capillary basement membranes.

Repeated low-dose challenge studies. Low transmission frequency per sexual contact in humans suggests that high-dose intravenous challenge (>300 TCID50 in a SHIV model) is an unrealistic way to evaluate prophylaxis. Repeated low-dose viral challenge (3-10 TCID₅₀) is an established means of modeling human exposure to virus (Hessell et al., Nat. Med., 15:951 (2009a) and Hessell et al., PLoS Pathog., 5:e1000433 (2009b)). Accordingly, rhesus macaques are initially challenged intrarectally with increasing doses of SHIV-SF162P3, beginning at 3 TCID₅₀, and increasing until virus is detected in all four control animals. The final dose is expected to be in the 10-20 TCID₅₀ range. The final dose challenge is continued every three days, for six months (60 maximum challenges) or until every animal has been infected. A Kaplan-Meier analysis of uninfected animals is performed, and a log-rank (Mantel Cox) test is used to assess statistical significance. Viral loads and T-cell counts are assessed, and biweekly plasma viral RNA load measurements are performed. Macaques bearing MHC alleles (Mamu-A*01, Mamu-B*08, Mamu-B*17) known to be protective from SIV challenge are excluded, to minimize MHC-associated variation.

SHIV-SF162P3, an R5 SHIV, is selected because the question here is prophylaxis from mucosal challenge, especially in the presence of anti-inhibitor antibodies. Because transmission is mediated by CCR5, SHIV-SF162P3, is more likely to provide relevant results than would SHIV-89.6P (Harouse et al., J. Virol., 75:1990 (2001) and Pahar et al., Virology, 363:36 (2007)). Moreover, two recent studies of passive antibody protection used the SHIV-SF162P3 with repeated low dose challenges (Hessell et al., 2009a; and Hessell et al., 2009b).

All references cited herein are fully incorporated by reference. Having now fully described the invention, it will be understood by those of skill in the art that the invention may be performed within a wide and equivalent range of conditions, parameters and the like, without affecting the spirit or scope of the invention or any embodiment thereof.

1. A therapeutic composition comprising an amount of a nucleic acid molecule encoding a fusion polypeptide comprising a CCR5 mimetic peptide, a soluble CD4 polypeptide that binds HIV gp120 and a Fc binding region of an immunoglobulin.

2. The composition of claim 1 wherein the soluble CD4 polypeptide is a variant CD4 polypeptide.

3. The composition of claim 2 wherein the variant CD4 polypeptide includes a substitution at position 40, 45 or 63.

4. The composition of claim 3 wherein the substitution is a Q to A, N, S, T, G, L, or I.

5. The composition of claim 1 or 2 wherein the fusion polypeptide has enhanced affinity for HIV gp120 relative to a fusion protein having sequences corresponding to wild-type CD4 and the Fc binding region.

6. The composition of claim 1 or 2 wherein the fusion polypeptide has enhanced affinity for CCR5 relative to a fusion protein having sequences corresponding to the CCR5 mimetic peptide and the Fc binding region.

7. The composition of claim 1 wherein the CCR5 mimetic peptide is from 10 to 35 amino acids in length and includes X₁X₂(Y)X₃X₄(Y)X₅X₆X₇(Y)(Y)X₈X₉X₁₀X₁₁ (SEQ ID NO:1), wherein X₁-X₂ and X₈-X₁₁ are independently absent or independently D, N, Q, H, G, Y, M, K, T, S, E, P or sY, wherein sY indicates a sulfated tyrosine, wherein X₃-X₄ and X₅-X₇ are independently D, N, Q, H, G, Y, M, K, T, S, E, P or sY, and wherein each (Y) may be sulfated.

8. The composition of claim 1 wherein the CCR5 mimetic peptide is from 10 to 35 amino acids in length and includes X₁X₂YX₃X₄YX₅X₆X₇YYYX₈ (SEQ ID NO:2), wherein X₁ is absent or is G, A, L, or I; X₂, X₄ and X₅ are independently D, N, Q, H, or E; X₃, X₆ and X₇ are independently G, A, L, or I; and X₈ is D, N, H, Q, or E.

9. The composition of claim 8 wherein X₂, X₄ or X₅ is D.

10. The composition of claim 1 wherein the CCR5 mimetic peptide has GDYADYDGGYYYD (SEQ ID NO:12), GDYADYDGGYYYDM (SEQ ID NO:13), GDYADYDGGYYYDG (SEQ ID NO:14), DYADYDGGYYYDMD (SEQ ID NO:4), DYADYDGGYYYDMDG (SEQ ID NO:17 ), DYADYDGGYYYD (SEQ ID NO:16) or DYADYDGGYYYDMDGG (SEQ ID NO:6).

11. The composition of claim 1 wherein the CD4 polypeptide has A, N, S, T, G, L, or I at position 40, 45 or 63.

12. The composition of claim 1 wherein the CCR5 mimetic peptide is C-terminal to the Fc binding region.

13. The composition of claim 1 wherein the CCR5 mimetic peptide is N-terminal to the soluble CD4 polypeptide.

14. The composition of claim 1 wherein the CCR5 mimetic peptide has at least 90% amino acid sequence identity to one of SEQ ID Nos. 2-16 or 18.

15. The composition of claim 1 wherein the CD4 polypeptide has at least 90% amino acid sequence identity to SEQ ID NO:20.

16. The composition of claim 1 wherein the Fc binding region has at least 90% amino acid sequence identity to SEQ ID NO:18.

17. A recombinant virus comprising a nucleic acid molecule encoding a fusion polypeptide comprising a CCR5 mimetic peptide, a soluble CD4 polypeptide that binds HIV gp120 and a Fc binding region of an immunoglobulin.

18. The recombinant virus of claim 17 which is an adeno-associated virus (AAV).

19. A method to prevent, inhibit or treat HIV infection in a mammal, comprising administering to a mammal in need thereof an effective amount of the composition of any one of claims 1 to 16 or the recombinant virus of claim 17 or 18.

20. The method of claim 19 wherein the composition is administered intramuscularly.

21. The method of claim 19 wherein the composition is mucosally administered.

22. The method of claim 19 wherein the composition is injected.

23. The method of claim 19 wherein the composition is intravenously administered.
